(19) United States
(12) Patent Application Publication
(10) Pub. No.: US 2003/0003508 A1
(43) Pub. Date: Jan. 2, 2003

(54) Serological diagnostic method with rickettsia pulicis bacterium

(76) Inventors: Didier Raoult, Marseille (FR); Bernard La Scola, Rousset (FR)

Correspondence Address: ANTONELLI TERRY STOUT AND KRAUS, SUITE 1800, 1300 NORTH SEVENTEENTH STREET, ARLINGTON, VA, 22209

(21) Appl. No.: 10/149,598

(22) PCT Filed: Dec. 12, 2000
(86) PCT No.: PCT/FR00/03475

(30) Foreign Application Priority Data
Dec. 14, 1999 (FR) 99/15777
Dec. 14, 1999 (FR) 99/15777
Dec. 14, 1999 (FR) 99/15777

(51) Int. Cl. G01N 33/53

(52) U.S. Cl. 435/7.1

(57) ABSTRACT

The invention concerns a method for isolating Rickettsia pulicis bacterium on clawed frog cells or any other cell at a culture temperature of 28°. The bacterium is used for a serological diagnostic method which consists in contacting the bacterium responsible for said disease with the patient's serum or biological fluid. The invention also concerns a device for implementing said method for in vitro detection of the bacterium in fleas or human samples.

This invention concerns the field of microbiology, notably the field of microbiological diagnosis. More precisely, the invention concerns a method for isolating a new bacterium temporarily called “Rickettsia pulicis” as well as using the strain to carry out serological testing.

The invention thus concerns a method for in vitro serological diagnosis of infections caused by “Rickettsia pulicis” as well as a device for implementing this method. The invention also concerns a kit for in vitro detection of the bacterium.

In the United States, a bacterium has been found but not isolated from fleas through indirect staining reactions using the Gimenez method which stain Rickettsias by means of antibodies directed against Rickettsias and by detection of gene sequences specific to Rickettsias for this bacterium. At the time, it was named ELB agent [Adams J R., Am. J. Trop. Med Hyg., 1990; 43:400-409). Genes common to the Rickettsias were amplified in this bacterium and sequenced, showing that the bacterium was a new one. This bacterium is regarded as one factor explaining the prevalence of flea-borne Rickettsia in California [Schriefer M E., J. Med. Entomol., 1994; 31:681-5]. One case [Schriefer M E., J. Clin. Microbiol., 1994; 32:949-54] was reported of infection in humans related to this bacterium. To date, many attempts have been made to culture this bacterium but always unsuccessfully. Cultures were also attempted from animals (VERO cells and L229 cell) [Higgins J A., J. Clin. Microbiol., 1996; 34:671-4; Radulovic S., Infect. Immun., 1995; 63:4826-9; Radulovic S., Antimicrobiol. Agent. Chemother., 1995; 39:2564-6]. The above three publications reported a culture of the bacterium in question which was named Rickettsia felis. However, these publications were not confirmed and no strain is available in the laboratories which claim to have isolated this bacterium. Samples sent by these laboratories were either samples devoid of any rickettsia or samples contaminated by another rickettsia, Rickettsia typhi. SDS-PAGE analysis shows that the strain isolated by these authors, named R. felis, was in fact R. typhi and, to all intents and purposes, did not correspond to the ELB agent. This is why the bacterium according to this invention, which is in fact the first isolate of the ELB agent, has been temporarily called “Rickettsia pulicis”.

Therefore, prior to this invention, there was no possibility of obtaining a serological reaction and, moreover, no isolate of this bacterium was available in any collection or laboratory. On the other hand, many fleas are naturally infected and there are flea collections which are 100% infected as a result of systematic transmission of this bacteria from mother to descendents.

The inventors have developed a specific and original method for culturing the “Rickettsia pulicis” bacterium.

Bacteria of the Rickettsia genus are usually cultured at 37° C. in the case of bacteria of the typhus group and at 32° C. for bacteria of the spotted-fever group [Weiss E., Annu. Rev. Microbiol., 1982; 36:345-70].

Attempts at culturing mammalian VERO L929, HEL and MRC5 cells or bird cells at 37° C. and 32° C. have not been successful.

The inventors have surprisingly discovered that culturing host cells must be carried out at a temperature below 30° C., preferably between 25° C. and 30° C., and still more preferably at 28° C.

The inventors carried out inoculation of ground fleas on a Xenopus laevis XTC-2 cell line cultured at 28° C. The bacterium is obtained 14 to 28 days later. 19/20 fleas which tested positive by genome detection (PCR) were successfully cultured by means of this method. This constitutes a 95% success ratio. The reference bacterium isolated (URRWFXCal2) was established and 10 passages have currently been obtained. The bacterium isolated has the genetic characteristics described for the ELB agent with regard to citrate synthase genes and RNA 16S (Gen Bank U 33922 and L28944 respectively).

This invention therefore concerns the Rickettsia pulicis bacterium isolated in this way and which has been established in culture as a source of antigens.

The term “established in culture” means that the bacterium is obtained in a reproducible manner and multiplies in time after successive re-inoculation of cell cultures.

More particularly, this invention covers a Rickettsia pulicis bacterium isolated and established on a cell culture-to-culture at a temperature of 25 to 30° C., preferably at 28° C.

More particularly still, the Rickettsia pulicis bacterium according to the invention is cultured on Xenopus laevis cells, line XTC-2.

In one embodiment, the isolation method according to the invention consists of the following steps:

1) a cell culture is obtained, preferably a Xenopus laevis XTC-2 cell line culture, at a culture temperature between 25 and 30° C., preferably at 28° C., and

2) bacteria are inoculated by centrifugation of ground fleas, or any other sample from a human or animal source infected by said bacteria, on a layer of the cells in culture from step 1, and incubated between 25 and 30° C., preferable at 28° C., until bacterial growth is observed.

3) bacteria are established in culture by successive re-inoculations on cultured cells.

The bacteria can be purified by centrifugation of the supernatant of the cell culture described in step 3 above.

This invention also concerns an antigen of said bacterium, in particular a protein chosen from the 17 Kd, 30 Kd and 150 Kd proteins, determined by means of polyacrylamide gel electrophoresis using the SDS-PAGE technique.

The invention also concerns a specific antibody against the bacterium according to the invention. More particularly, a polyclonal antibody of animal origin, notably a mouse or rabbit immunoglobulin.

This invention also concerns the detection of a specific antibody to a human immunoglobulin which recognizes said bacterium, preferably IgG, IgM or IgA, and more particularly an animal immunoglobulin, notably a goat anti-human immunoglobulin.

This invention also covers a cell culture of a bacterium according to the invention and more particularly Xenopus laevis cells, preferably XTC-2 cells.

The bacterium according to the invention was filed with CNCM (National Collection of Microorganism Cultures at the Institut Pasteur, France) on Dec. 8, 1999 under no. I-2363, identified as Rickettsia pulicis (URRWFX cal 2) in the form of a cell culture of infected XTC-2 cells. A culture of healthy XTC-2 cells (not infected) was filed with CNCM on Dec. 8, 1999 under no. I-2364.

This invention also covers the use of a bacterium, culture or specific antibody according to the invention in an in vitro diagnostic method for diseases related to infection by the Rickettsia pulicis bacterium, as well as a method for serological diagnosis of infection by Rickettsia pulicis bacterium according to the invention. This method consists in contacting serum or any other biological fluid from a patient with said bacterium and detecting an immunological reaction.

More particularly, this invention covers an in vitro method for serological diagnosis of Rickettsia pulicis infections in which the bacterium according to the invention, a culture according to the invention or a specific antibody according to the invention is contacted with a sample from the patient consisting of serum, biological fluid, fleas or human sample.

The method according to the invention includes a step which basically consists of detecting an immunological reaction between an antibody specific to the bacterium according to the invention and an antigen to said bacterium, or between a specific antibody of an immunoglobulin according to the invention which recognizes said bacterium and said human immunoglobulin which recognizes said bacterium.

In one embodiment, the diagnostic method according to the invention consists of:

depositing a solution of the bacterium according to the invention in or onto a solid support, notably 0.5 to 5 μl, preferably 1 μl of said solution containing said bacterium,

adding dilute serum or biological fluid to be tested in or onto said support,

adding a labelled antibody solution in or onto the support, notably an animal anti-human immunoglobulin specific to human immunoglobulins of the IgG, IgM or IgA type which recognizes said bacterium,

incubating for a fixed period of time,

rinsing the solid support if necessary and

carrying out detection of the immunological reaction between a human antibody recognizing said bacterium and said anti-human immunoglobulin.

Any device suitable for cell and bacterial suspensions can be used as a solid support, especially tubes, glass slides, Bijoux-type tubes or rigid polyethylene, polystyrene, polyvinyl chloride or nitrocellulose microtitration plates with microwells, glass slides being the preferred medium.

The human antibody detected is an immunoglobulin, notably of the G, M or A type, specific to the bacterium according to the invention.

Advantageously, the diagnostic method according to the invention uses an ELISA type immunoenzyme assay or an immunoflourescent assay.

Enzymatic, radioactive or fluorescent labelling is thus preferred as labelling for the anti-human immunoglobulin, fluorescent labelling being the preferred method.

The term “fluorescent labelling” means the antibody is made fluorescent by coupling or forming a complex with a suitable fluorescent agent such as fluoresceine iso(thio)cyanate.

The term “radioactive labelling” means the antibody carries a radioactive isotope allowing the assay to be carried out by a radioactivity count, the isotope being carried either on one element of the antibody structure, for example constitutive tyrosine residues, or on an appropriate radical attached to it.

The term “enzymatic labelling” means the specific antibody is coupled to an enzyme which, combined with the use of suitable reagents, allows the specific antibody to be quantitatively measured.

The substrate and reagents are selected such that the final product of the reaction or sequence of reactions triggered by the enzyme and using these substances is:

either a stained or fluorescent substance which diffuses into the neighbouring liquid medium of the sample tested and is either measured by spectrophotometry or fluorimetry or by visual evaluation and compared to a standard colour range if necessary,

or an insoluble or fluorescent stained substance which becomes deposited on the sample tested and which can be measured by reflection photometry or by visual evaluation and compared to a standard colour range if necessary.

When a fluorescent antibody is used, the fluorescence of the sample tested is read directly on a suitable apparatus.

When a radioactive probe is used, for example iodine 125, radioactivity associated with the sample tested is counted in a gamma counter using any suitable method and, for example, after dissolving the cells in an alkaline solution (e.g. a soda solution) and recovering the solution containing the radioactivity by means of an absorbent buffer.

When an enzyme attached to a specific antibody is used, the stained or fluorescent product is obtained by adding a solution containing the enzyme substrate and one or more additional agents which result in a final product that is either a stained product soluble in the medium, an insoluble stained product or a soluble fluorescent product, as explained above. Next, the light signal is measured using a device adapted to each situation: transmission photometer, reflection photometer or fluorimeter.

Alternatively, the colour produced can also be evaluated visually, using a standard coloured solution range of need be.

When an alkaline phosphatase is used as the enzyme, this enzyme is coupled to the specific antibody according to the Boehringer Mannheim-Biochemica method. The preferred substrates for this enzyme are paranitrophenylphosphate for fluorimetric reading or 5-bromo-4-chloro-umbelliferyl phosphate for fluorimetric reading or 5-bromo-4-indolyl-6-phosphate to obtain an insoluble stained reaction product. β-galactosidase can also be used as the enzyme, for which the preferred substrates are orthonitrophenyl β-D-galactopyranoside or 4-methyl-umbelliferyl β-D-galactopyranoside.

Preferentially, the specific antibodies can be coupled to peroxidase. In this case, the coupling process is based on the technique described by M. B. Wilson and P. K. Nakane in Immunofluorescence and related staining techniques, W. Knapp, K. Kolubar, G. Wicks ed. Elsevier/North Holland, Amsterdam, 1978, p. 215-224.

The reagents used to show the presence of peroxidase conjugated to specific antibodies contain oxygenated water, the enzyme substrate and a suitable chromogen, for example orthophenylenediamine or 2,2′bis-azino (3-ethyl thiazoline sulphonic) acid or ABTS to obtain a stained and soluble final reaction product, or 3,3′-diamino benzidine or 3-amino-9-ethyl carbazole or 4-chloro-α-naphthol to obtain an insoluble final reaction product, or parahydroxyphenyl propionic acid to obtain a fluorescent reaction product soluble in the medium.

Another embodiment of the invention is the use of specific antibodies coupled to acetylcholinesterase.

Acetylcholinesterase is coupled to the antibody preferably by means of a process based on that described in French patent no. 2 550 799 or a process which includes preparation of antibody fragments using a known technique, modification of the enzyme by reaction with a suitable heterobifunctional agent followed by coupling the products obtained in this way. Other known processes for constructing conjugated immunoenzymes can also be used in this case.

Detection of enzyme activity specifically related to the antigen recognized by acetylcholinesterase is preferably based on the well-established technique using acetylthiocholine as an enzyme substrate and Ellman reagent or 5,5′-dithio-2-nitro-benzoic acid as the chromogen, according to any variant adapted to the case in question, for example that described by Pradelles et al, Anal. Chem., 1985; 57:1170-1173.

The chromogens mentioned are used in their natural form or in the form of water-soluble salts.

The serological diagnostic method according to the invention is adapted to use in biology and/or anatomopatholgy laboratories. To this end, we propose a device for implementing this method which includes a solid support onto or into which is deposited a solution containing the bacterium according to the invention, as defined earlier.

Another aspect of the invention also concerns a kit for in vitro detection of Rickettsia pulicis. This kit consists of the following components:

a solution containing Rickettsia pulicis according to the invention, isolated and established as described earlier, as the positive control,

a solution containing a specific antibody recognizing the bacterium according to the invention and/or a solution containing a specific antibody of a human immunoglobulin which recognizes the bacterium according to the invention, preferably labelled,

possibly a rinsing solution.

The specific antibody used in the kit according to the invention is advantageously labelled with a radioactive probe, an enzyme or a fluorescent agent.

Where the specific antibody is labelled with an enzyme, the kit also includes an enzyme substrate and one or more reagents to allow enzyme activity to be seen with the naked eye.

Where the specific antibody is labelled with a fluorescent agent, fluoresceine iso(thio)cyanate is preferred.

According to a preferred embodiment of the invention, an immunoglobulin, particularly mouse immunoglobulin, is used as the specific antibody.

The invention will become clearer on reading the description below, divided into sections, which describes experiments conducted with the aim of applying the invention. These examples are given purely for the purpose of illustration.

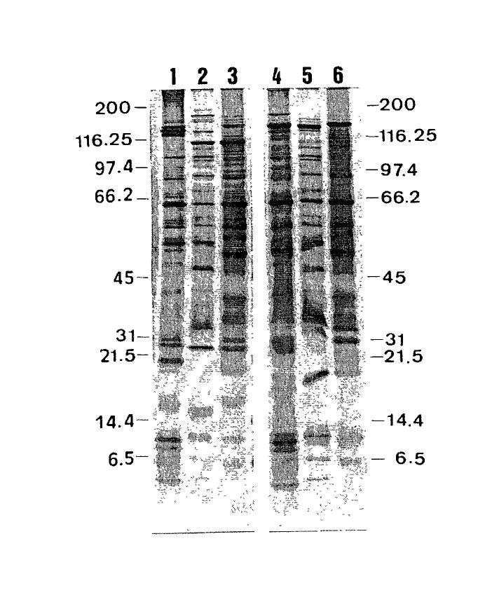

FIG. 1 represents the protein profile of R. conorii, R. pulicis and R. typhi bacteria tested in example 4.

### EXAMPLE 1

### Procedure for Isolating R. pulicis on XTC Cells.

1-Primo-isolation. Primo-isolation was carried out using a centrifugation technique in Bijoux tubes inoculated with a Xenopus laevis cell line [Pudney M, Varma M R G, Leake C J. Establishment of a cell line (XTC-2) from the south African clawed toad Xenopus laevis. Experimentia. 29:466-467]. These cells are cultured on Leibowitz-15 medium with L-glutamine and L-amino acids (Gibco, Gaithersburg, Md.) to which 5% foetal calf serum (Gibco) and 2% tryptose phosphate (Gibco) are added. The Bijoux tubes (Sterilin-Felthan-England, 3.7 ml) including a 12 mm support coverslip are inoculated with 1 ml of culture medium containing about 50 000 cells and incubated at 28° C. for 24 to 48 hours such that a layer of subconfluent cells is obtained. Isolation of bacteria was attempted on 2 batches of cat fleas (Ctenocephalides felis). Each batch consisted of 50 fleas. Fleas were grouped together in 20 groups of 5 fleas each (California group 1 to 10 and Pete 1 to 10). In each group, fleas were decontaminated by immersion in 70% methyl alcohol containing 0.2% iodine for 5 minutes. Fleas were then rinsed in distilled water, frozen in liquid nitrogen and ground. Ground fleas were then taken up in 0.8 ml of culture medium and the powder was used to inoculate Bijoux tubes (1 per powder). These tubes were then centrifuged at 700×g for 1 hour at 22° C. The supernatant was withdrawn, the layer was washed twice in sterile PBS buffer then incubated at 28° C. with 1 ml of medium to which was added cotrimoxazole at a final concentration of 4 μg/ml. Each week, the supernatant was replaced by fresh culture medium. The supernatant removed was cryocentrifuged for Gimenez staining [Gimenez DF. 1964. Staining rickettsiae in yolk-sac cultures. Stain Technol. 39:135-140]. Where the Gimenez stain allowed intracellular bacteria to be detected (between D15 and D30 after inoculation), cells were detached from the coverslip and inoculated onto a subconfluent cell layer using a 25-cm² culture dish with 5 ml of culture medium then incubated at 28° C. The supernatant was used to detect Rickettsia sp. by amplification and sequencing of the citrate synthase gene. With the exception of the California 1 group, all other groups were positive.

2- Propagation of the isolate. The rate of infection in cell culture dishes was checked every 2 days by carrying out Gimenez staining of infected cells in the supernatant. When the rate of infection was at a maximum, cell cultures were re-inoculated with 30 ml of culture medium on 170 cm² culture dishes containing healthy subconfluent XTC-2 cells. . For mass production of the California 2 strain (URRWFXCal2), we used an infected cell multiplication technique by trypsination. Five to six days after infection of XTC-2 cells in a 174 cm² dish, when the rate of cell infection was over 80%, the culture supernatant was removed, the cell layer was washed with Rinaldini then incubated for 5 minutes at 37“C with 0.05% trypsine (Gibco). Infected cells were resuspended in 150 ml of culture medium. Infected cells diluted in this way were divided into 5×174 cm² culture dishes at a rate of 30 ml per dish. The same procedure was repeated for each dish every 5 days in order to produce the equivalent of 200 of these 174 cm² dishes in order to obtain a sufficient quantity of material for later studies. The absence of contaminants in the cell cultures was checked throughout the production procedure by systematic culturing of the culture dishes on blood gelose and soya trypticase gelose.

3-Electron microscopy. Infected cells, inoculated with bacteria 8 days before, were collected then prepared for electron microscopy. Cells were fixed in a solution of 2.5% glutaraldehyde in 0.15 M PBS buffer (Biomérieux, Marcy l'étoile, France) for 1 hour at 4° C. Cells were rinsed overnight in the same buffer then fixed for 1 hour at room temperature in osmium tetroxide in 0.15 M PBS buffer. Dehydration was carried out by successive rinsing in increasingly concentrated acetone solutions (Carlo Erba, Val de Reuil, France). Cells were then fixed in Araldite boxes (Fluka, St Quentin Fallavier, France). Thin sections were cut using an Ultracut microtome (Reicher-Leica, Marseille, France) then stained with a saturated solution of uranyl acetate (Merck, Damstadt, Germany) in methanol, lead nitrate and sodium citrate (Merck) in water prior to examination under a Jeol 1220 electron microscope (Jeol, Croissy sur Seine, France). This examination made it possible to observe bacteria in intracellular localisations or free in the cytoplasm. No bacteria were found in the nucleus.

### EXAMPLE 2

### Production and Characterisation of Mouse Polyclonal Antibodies.

The bacteria used to inoculate mice were first purified using 174 cm² cell culture dishes infected at a rate of 80%. The supernatant was withdrawn from the dishes and cells were incubated for 45 minutes at 30° C. with 0.5% trypsine (Gibco). Cells were taken up with the supernatant then lysed by 6 sonication steps (40 watts −1 min.). Residual cells were removed by centrifugation at 1500 rpm for 15 minutes. The supernatant was deposited on a 25% sucrose solution in PBS. After centrifugation for 30 minutes at 7500 rpm at 4° C., the residue containing the bacteria was taken up in 2 ml of PBS. Bacteria were finally purified by centrifugation at 25 000 rpm for 1 hour at 4° C. in a Renografine gradient (45 to 25%). After centrifugation, the layer corresponding to the bacteria was collected and bacteria were rinsed in PBS by means of 2 centrifugation steps at 10 000 rpm for 10 minutes.

Mice aged between 6 and 8 weeks were inoculated by intraperitoneal route at D0, D10, D20 and D30 with 0.5 ml of a 10⁶/ml solution of purified bacteria and Freund's complete adjuvant. At D40, mice were sacrificed and blood collected by intracardiac puncture. After separation, the serum was tested by indirect immunofluorescence then frozen at −20“C. Serum was diluted to 1:50 and adsorbed with XTC-2 cells prior to use in order to withdraw antimouse antibodies.

### EXAMPLE 3

### Serodiagnosis by Indirect Immunoflourescence and Western Blot

1- Preparation of the Antigen

Rickettsia pulicis (URRWFXCal2) was cultured on confluent layers of XTC-2 cells as described previously. R. conorii (Moroccan strain, ATC VR 141), R. prowazekii (Brein-1) and R. typhi (Wilmington strain, ATCC VR 144) were cultured on confluent layers Vero cells in 174 cm² culture dishes, at 32° C., 35° C. and 35° C. respectively. When 80% of cells were infected, cells and the supernatant were collected, centrifuged at 10 000×g for 10 minutes, washed 4 times on 40 ml of PBS buffer, then resuspended in the smallest possible volume of sterile distilled water. The final protein content of this solution was measured by means of ultraviolet spectrophotometry then adjusted to a final concentration of 1 mg/ml before being frozen at −20° C.

2- Indirect Immunofluorescence (IFA)

The 4 antigens were then deposited in each of the wells of a 30-well slide (Dynatech Laboratories Ltd., Billingshurt, United Kingdom), dried in air then fixed in acetone for 10 minutes. All sera were diluted to ¼, ⅛, 1/16, 1/32, 1/64 and 1/128 in PBS with 3% skimmed milk powder. Indirect immunofluorescence with determination of IgG and IgM after adsorption of the rheumatoid factor was carried out using a standard laboratory technique [La Scola, B. et aL. Laboratory diagnosis of rickettsioses: current approaches to diagnosis of old and new rickettsial diseases. (1997). J. Clin. Microbiol. 35:2715-2727]. Titres of 1/64 IgG and/or 1/32 IgM were considered to be positive. In the case of a cross serological reaction between several rickettsia species, a serological reaction was considered to be specifically directed against a species if the sum of IgG and IgM titres was at least two dilutions higher than the IgG+IgM titres against the species for which the cross reaction was observed.

3-Western Blot

After purification, R. pulicis, R. typhi and R. conorii were suspended in sterile distilled water and the concentration adjusted to 2 mg/ml. The procedure used to carry out a Western blot has been described before [Eremeeva M. N. et al. Serological response of patients suffering from primary and recrudescent typhus: comparison of complement fixation reaction, Weil-Felix test, microimmunofluorescence and immunoblotting. Clin. Diaf. Lab. Immunol. (1995). 1:318-324]. Antibodies reacting against 20-50 kD antigens are specific to LPS in R. typhi and R. conorii. The intensity of various reaction bands was evaluated by video imaging (The Imager, Appligene, Illkirch, France).

4-Choice of Serum

In order to evaluate the seroprevalence of R. pulicis in the general population, 100 sera from blood donors was tested. We tested serum in the acute phase from 97 patients with spotted fever, 16 with murine typhus and 67 with epidemic typhus in order to test for serological cross reactions between R. conorii and R. typhi. We therefore have clinical and epidemiological data for this set of patients. All these sera were tested for R. pulicis, R. prowazekii, R. typhi and R. conorii.

5-Serology Results

None of the 100 sera from blood donors showed positive serology against R. pulicis.

A serological cross reaction between R. prowazekii and R. pulicis was observed in 51 of the 67 sera from patients with epidemic typhus (76.1%). However, none of the 67 sera had an anti-R. pulicis antibody titre higher than the anti-R. prowazekii antibody titre, be it for IgG or IgM.

A serological cross reaction between R. typhi and R. pulicis was observed in 11 of the 16 sera from patients with murine typhus (68.7%). However, none of the 16 sera had an anti-R. pulicis antibody titre higher than the anti-R. typhi antibody titre, be it for IgG or IgM.

A serological cross reaction between R. conorii and R. pulicis was observed in 67 of the 97 sera from patients with Mediterranean spotted fever (69.0%). 96 of the 97 sera had anti-R. pulicis antibody titres that were lower than anti-R. conorri antibody titres, be it for IgG or IgM. One patient presented a 2-dilution higher anti-R. pulicis antibody titre than that against R. conorii and was consequently suspected of being infected by R. pulicis. A Western Blot test was carried out on this serum and confirmed that the patient carried antibodies specifically directed against R. pulicis.

### EXAMPLE 4

4.1 Analysis of Principal Proteins by Polyacrylamide-sodium Dodecylsulphate Gel Electrophoresis (SDS-PAGE) and Western Transfer.

Rickettsia strains were purified with Renografine. Protein preparations of R, pulicis, R. typhi, R. prowazekii, R. canadensis, R. conorii and R. akari purified with Renografine were placed in suspension in an SDS-PAGE sample buffer (Tris 0.625 M, pH 8.0, 2% (w/v) sodium dodecylsulphate, 5% (v/v) 2-mercaptoethanol, 10% (v/v) glycerol and 0.002% (w/v) bromophenol blue). One aliquot of each of these was heated at 100° C. for 10 minutes then heated and unheated aliquots were loaded onto a polyacrylamide gel linear gradient of 9-16% (18 cm×20 cm×1.5 mm). Proteins were then separated by electrophoresis at 40 mA for 5 hours at 10° C. (Laemmli, 1970). Separated proteins were stained with silver. The principal immunogenic proteins were studied by Western transfer using unheated antigens purified as described previously (Laemmli, 1970; Teysseire et al., 1992) and anti-R. pulicis murine polyclonal antibodies.

4.2 Sensitivity to Antibodies

Erythromycin activity against R. pulicis was determined in Vero cells by colorimetric assay using Gimenez staining. Vero cell cultures on titration microplates with 48 wells were infected with 2000 PFU rickettsia for 1 hour at room temperature and erythromycin was added at different concentrations in different rows. Rows without the drug infected with 2000, 200, 20 and 0 PFU acted as controls. After 9 days of plate incubation at 32° C., cell culture monolayers were stained with a Gimenez stain to reveal the presence of infected sites (collections of rickettsia) in the 25 sites chosen at random for each well. The minimum antibiotic concentration leading to complete inhibition of infected sites with respect to the antibiotic-free control were marked as MIC. For doxycycline and rifampin, only one concentration, 4 μg/ml, was tested. The experiments were carried out in double to confirm the results. The incubation temperature was lowered to 32° C. to allow R. pulicis to grow. R. conorii and R. typhi were treated in the same way as controls.

4.3 Results and Discussion

FIG. 1 shows the silver stained SDS-PAGE profile of protein preparations of whole cells. Line 1: R. conorii; Line 2: R. Pulicis, Line 3: R. typhi, Line 4: R. conorii, Line 5: R. pulicis, Line 6: R. akari. Molecular weight standards appear at the extremities of the gel.

Lines 1 to 3 correspond to cold antigens. Lines 4 to 6 correspond to hot antigens.

The SDS-PAGE profile of R. pulicis differs substantially from those obtained for R. typhi (FIG. 1), R. prowazekii, R. canadensis, R. conorii (FIG. 2) and R. akari. R. pulicis, in the same way as R. conorii, has a high molecular weight protein with a MW of over 150 kDa, which is not the case for R. typhi and R. prowazekii. R. pulicis also has a thermolabile protein of 30 kDa which was not found in the profiles of the other rickettsias studied. The SDS-PAGE protein profile of the strain according to this invention is therefore considerably different from that of “R. felis” which, as was mentioned earlier, closely resembles that of R. typhi in terms of Western transfer (Radulovic et al. 1995b; Higgins et al. 1996; Azad et al. 1997) whereas it has been shown that the immunogenic antigens of R. pulicis are distinguishable from those of R. typhi in human serum reacting specifically to R. pulicis and in murine antisera produced against the purified isolate as a result of a reaction against the 30 kDa antigen.

Fluorescence and and dual staining of actin and bacteria was carried out in order to evaluate the ability of R. pulicis to polymerise actin intracellularly. Polar polymerisation of actin was not observed in cells infected with R. pulicis or R. typhi, contrary to cells infected with R. conorii. It would seem that actin polymerisation is associated with the capacity to grow inside the nucleus, a property common to all the rickettsias of the spotted fever group studied so far but absent in the typhus and R. pulicis group (Heinzen et al., 1993; Teysseire et al., 1992; Burgdorfer et al., 1968).

The MIC for erythromycin with R. pulicis was 32 pg/ml and a concentration of 4 pg/ml of doxycycline or rifampin was inhibitory. The results obtained for the controls were identical to those obtained previously (Rolain et al., 1998). The R. pulicis isolate is resistant to erythromycin while sensitivity to this antibiotic is a characteristic of rickettsias of the typhus group (Rolain et al., 1998) and the bacterium known as “R. felis” ((Radulovic et al. 1995a; Radulovic et al. 1996).

The rOmpA and rpoB genes were sequenced from base 3539 to base 6722 and from base 1 to base 3866 respectively. For rpoB, sequence similarity was from 96% (R. massiliae, bar 29, R. conorii) to 87% for nucleotide sequences. Sequence similarity was 92% between R. pulicis and R. prowazekii. With regard to amino acid sequences, similarity was 98% (R. massiliae, Bar 29, R. conorii) to 83% (R. typhi). Sequence similarity was 96% between R. pulicis and R. prowazekii. The dendrograms obtained from rOmpA with the three different tree construction methods used showed a similar phylogenetic position for R. pulicis. They formed clusters with R. australis with a boot shrap value of 100%.

Additional data which might differentiate R. pulicis from the “R. felis” strain, now lost, are growth temperature. R. pulicis did not grow at 35 or 37° C. whereas it has been reported that “R. felis” causes a cytopathic effect leading to the formation of small delayed areas of growth when it is grown at 34° C. in Vero cells, HUVEC or L929 cells ((Radulovic et al. 1995b). What is more, R. pulicis, even at 28° C., does not have the ability to grow on L929 cells. These results demonstrate that the “R. felis” isolate, now lost, was not the same organism as that recovered repeatedly from infected cat fleas supplied by the El Laboratory. The phenotype description of the R. pulicis isolate is substantially different from that of the original “R. felis” isolate. Moreover, the inventors found they were only able to isolate R. typhi from stocks of the original “R. felis” strain which were sent to their laboratory after it was isolated.

| Characteristic | R. conorii | R. typhi | R. pulicis | “R. felis” |
|---|---|---|---|---|
| Growth possible at | | | | |
| 28° C. | + | + | + | NF |
| 32° C. | + | + | +(low) | NF |
| 34° C. | + | + | − | + |
| Growth possible on | | | | |
| XTC-2 cells | + | + | + | NF |
| Vero cells | + | + | + | + |
| L929 cells | + | + | − | + |
| Localisation in the | | | | |
| Cytoplasm | + | + | + | + |
| Nucleus | + | − | − | − |
| Actin polymerisation | + | − | − | NF |
| Inhibition by 4 μg/ml of | | | | |
| Doxycycline | + | + | + | + |
| Rifampin | + | + | + | + |
| Erythromycin | − | + | − | + |
| Proteins on SDS-PAGE | | | | |
| 150 kDa | + | − | + | − |
| 30 kDa | − | − | + | − |
| 17 kDa | + | + | + | + |
| Genes detected | | | | |
| OmpA | + | − | + | − |
| OmpB | + | + | + | + |

### BIBLIOGRAPHY

Adams, J. R., Schmidtmann, E. T ., & Azad, A. F. (1990). Infection of colonized cat fleas, Ctenocephalides felis (Bouché), with a rickettsia-like microorganism. Am J Trop Med Hyg 43,400-409.

Azad, A. F., Sacci, J. B., Nelson, W. M., Dasch, G. A., Schmjdtmann, E. T., & Cari, M. (1992). Genetic characterizatjon and transovarial transmission of a typhus-like rickettsia found in cat fleas. Proc Natl Acad Sci USA 89, 43-46.

Azad, A. F., Radulovic, S., Higgins, j. A., Noden, B. H., & Troyer, J. M. (1997). Flea-borne rickettsioses: ecologic considerations. Emerg Infect Dis 3, 319-327.

I Bouyer, D. H., Crocquet-Valdes, P. A., & Walker, D. H. (2000). Expression and size I′ determination of the rOmpA protein of Rickettsia felis. In American Society for 5 Rickettsiology-15th meeting. pp. 61-61.

Burgdorfer, W., Anacker, R. L., Bird, R. G., & Bertram, D. S. (1968). Intranuclear growth of Rickettsia rickettsii. J Bacteriol 96,1415-1418.

Drancourt, M., Raoult, D. (1999). Characterization of mutations in rpoB gene in naturally rifampin-resistant Rickettsia species. Antimicrob Agents Chemother 43, 10 2400-2403.

Fournier, P. E., Roux, V., Raoult, D. (1998). Phylogenetic analysis of spotted fever group rickettsiae by study of the surface protein rOmpA. Int J Syst Bacteriol 48, 839-849.

Heinzen, R. A., Hayes, S. F., Peacock, M. G., & Hackstad, T. (1993). Directional ]3′, actin polymerization associated with spotted fever group ricketisia infection of Vero cells. Infect Immun 61, 1926-1935.

Higgins, J. A., Radulovic, S., Schriefer, M. E., & Azad, A. F. (1996). Rickettsia felis: a new species of pathogenic rickettsia isolated from cat fleas. J Cljn Microbiol 34,671-674.

20 La Scola, B., & Raoult, D. (1996). Diagnosis of Mediterranean spotted fever by cultivation of Rickettsia conorii from blood and skin samples using the centrifugation-shell vial technique and by detection of R. conorji in circulating

I.. endothelial cells: a 6 year follow-up. J Clin Microbiol 34, 2722-2727.

Laemmli, U. K. (1970). Cleavage of structural proteins dLiring the assembly of the 25 head of bacteriophage T4. Nature 227,680-685.

Murray, R. G. E., & Schleifer, K. H. (1994). Taxonomic notes: a proposai for recording the properties of putative taxa of procaryotes. Int J Syst Bacteriol 44,174-176.

Noden, B. H., Radulovic, S., Higgins, J. A., & Azad, A. F. (1998). Molecular 5 identification of Rickettsia typhi and R.felis in co-infected Ctenocephalides felis (Siphonaptera: Pulicidae). J Med Entomol 35, 410-414.

Radulovic, S., Higgins, J. A., Jaworski, D. C., & Azad, A. F. (1995a). In vitro and in vivo antibiotic susceptibilities of ELB rickettsiae. Antimicrob Agents Chemother

]0 Radulovic, S., Higgins, J. A., Jaworski, D. C., Oasch, G. A., & Azad, A. F. (1995b). Isolation, cultivation, and partial characterization of the ELB agent associated with cat fleas. Infect Immun 63, 4826-4829.

Radulovic, S., Higgins, J. A., Jaworski, O. C., & Azad, A. F. (1996). In vitro and in vivo antibiatic susceptibilities of ELB rickettsiae (val 39, pg 2564, 1996). Antimicrob.Agents Chemother., 40, 2912.

Ralain, J. M., Maurin, M., Vestris, G., & Raoult, 0. (1998). In vitro susceptibilities of 27 Rickettsiae to 13 antimicrobials. Antimicrob Agents Chemother 42,1537- 1541.

Teysseire, N., Chiche-Portiche, C., & Raoult, o. (1992). Intracellular mavements 20 of Rickettsia conorii and R. typhi based on actin palymerizatian; Res Microbiol 143, 821-829.

1. Rickettsia bacterium corresponding to the ELB agent, isolated and established in culture.

2. Bacterium according to claim 1 isolated in cell culture at a temperature of 25 to 30° C., preferably at 28° C.

3. Rickettsia bacterium, corresponding to the ELB agent, according to any of claims 1 or 2 obtained by a method in which
1) a cell culture is obtained, preferably a Xenopus laevis cells, at a culture temperature between 25 and 30° C., preferably at 28° C., and
2) bacteria are inoculated by centrifugation of ground fleas, or any other sample from a human or animal source infected by said bacteria, on a layer of the cells in culture from step 1, and incubated between 25 and 30° C., preferable at 28° C., until bacterial growth is observed,
3) bacteria are established in culture by successive re-inoculations on cultured cells.

4. Bacterium according to claim 3 wherein the bacteria are purified by centrifuging the supernatant of the cell culture in step 3.

5. Bacterium according to any one of claims 1 to 4 filed with CNCM (National Collection of Microorganism Cultures at the Institut Pasteur, France) on Dec. 8, 1999 under no. I-2363.

6. Antigen of a bacterium according to claims 1 to 5.

7. Antigen of a bacterium according to claim 6 wherein this is a protein chosen from the proteins with a molecular weight of 30 Kd and 150 Kd, determined by an SDS-PAGE technique.

8. Antigen produced from a bacterium according to claim 6 wherein it is a protein with a molecular weight of 17 kD determined by the SDS-PAGE technique.

9. Specific antibody against an antigen of the bacterium according to one of claims 1 to 8.

10. Antibody according to claim 9 wherein it is a polyclonal antibody of animal origin.

11. Antibody according to claim 10 wherein it is a mouse immunoglobulin.

12. Cell culture of a bacterium according to any one of claims 1 to 5.

13. Culture according to claim 12 wherein the cells are Xenopus laevis cells infected with said bacterium, preferably obtained from healthy XTC-2 cells, filed at the Institut Pasteur's CNCM on Dec. 8, 1999 under number I-2364.

14. Use of a bacterium according to any one of claims 1 to 5 or culture according to claims 12 or 13 for in vitro diagnosis of diseases related to infection by said bacterium.

15. Use of an antigen or antibody according to any one of claims 6 to 11 for in vitro diagnosis of diseases related to infection by bacteria according to any one of claims 1 to 5.

16. Method for in vitro serological diagnosis of Rickettsia infections that includes a step which basically consists in detecting an immunological reaction between a specific antibody of the bacterium according to one of claims 9 to 11 and an antigen of said bacterium.

17. Method for in vitro serological diagnosis of Rickettsia that includes a step which basically consists in detecting an immunological reaction between a specific antibody of a human immunoglobulin which recognizes said bacterium according to claims 1 to 5 and said human immunoglobulin which recognizes said bacterium according to claims 1 to 5.

18. Method according to claim 17 wherein the specific antibody of a human immunoglobulin, preferably of the G, M or A type, recognizing said Rickettsia bacterium according to one of claims 1 to 5 is an animal immunoglobulin, preferably a goat immunoglobulin.

19. Method for diagnosis of diseases related to infection by the Rickettsia bacterium according to claim 17 or 18 in which the bacterium according to any one of claims 1 to 5, a culture according to one of claims 12 or 13 and/or a specific antibody according to any one of claims 9 to 11 is contacted with a sample originating from a patient consisting of serum, biological fluid, fleas or a human sample.

20. Serological diagnostic method according to claim 19 which includes the following steps:
depositing a solution of the bacterium as defined in claims 1 to 5 in or onto a solid support,
adding the serum or biological fluid to be tested in or onto said support,
adding a specific labelled antibody solution of a human immunoglobulin which recognizes said bacterium in or onto the support
incubating for a fixed period of time,
rinsing the solid support and
carrying out detection of said immunological reaction.

21. Method according to claim 20 wherein the labelled antibody is labelled with a radioactive probe of an enzyme or fluorescent agent.

22. Method according to claim 21 wherein the fluorescent agent is fluroesceine isothiocyanate.

23. Method according to claims 20 to 22 wherein 0.5 to 5 μl, preferably about 1 μl, of said solution containing the bacterium is used.

24. Device for application of the method according to any one of claims 20 to 23 which includes a solid support into or onto which the solution containing the bacterium is deposited.

25. Device according to claim 24 wherein the solid support is a glass slide.

26. Kit for in vitro detection of the Rickettsia bacterium according to the method of one of claims 16 to 23 which consists of the following basic elements:
a solution containing the bacterium or cell culture as defined in claims 1 to 5 and 12-13, and/or
a solution containing a specific antibody as defined in claims 9 to 11, and/or
a solution containing a specific antibody of a human immunoglobulin which recognizes said bacterium according to claims 1 to 5.

27. Kit according to claim 26 wherein said specific antibody is labelled.

28. Kit according to claim 27 wherein said specific antibody is labelled with a radioactive isotope, an enzyme or a fluorescent compound.
